(19) United States
(12) Patent Application Publication
(10) Pub. No.: US 2026/0263876 A1
(43) Pub. Date: Sep. 10, 2026

(54) METHOD FOR RECORDING BREATHING EXERCISES PERFORMED ON A BREATHING EXERCISE DEVICE

(71) Applicant: CEGLA Medizintechnik GmbH, Montabaur (DE)

(72) Inventor: Andrea EBINGER, Montabaur (DE)

(73) Assignee: CEGLA Medizintechnik GmbH, Montabaur (DE)

(21) Appl. No.: 19/553,545

(22) Filed: Mar. 2, 2026

(30) Foreign Application Priority Data
Mar. 5, 2025 (DE) 10 2025 108 401.7

(51) Int. Cl. A63B 23/18; A63B 24/00

(52) U.S. Cl. CPC A63B 23/18; A63B 24/0062; A63B 2024/0068; A63B 2220/808; A63B 2220/833; A63B 2230/40

(57) ABSTRACT

To perform breathing exercises with a breathing exercise device, a breathing exercise device includes a housing with a flow channel in which a flow noise, influenced by the breathing air of a user, is generated and recordable with regard to intensity and/or vibration frequency profile and/or duration and/or amplitude using a data processing device. This flow noise is recorded by a microphone and forwarded to an evaluation unit, which analyzes it with regard to these parameters. The system gives the user real-time feedback on breathing technique and allows individual adjustments to the therapy. It also includes functions for recording breathing patterns, rest phases, and setting a variable or static stenosis in the flow channel. The method uses an electronic communicator and is integratable into the breathing exercise device, or into a smartphone, wearable, or other devices. It offers precise monitoring and evaluation of breathing exercises to maximize their effectiveness.

### CROSS REFERENCE TO RELATED APPLICATIONS

Applicant claims priority under 35 U.S.C. §119 of German Application No. 10 2025 108 401.7 filed Mar. 5, 2025, the disclosure of which is incorporated by reference.

### BACKGROUND OF THE INVENTION

### 1. Field of the Invention

The present invention relates to a method for recording breathing exercises performed on a breathing exercise device, wherein the breathing exercise device forms a flow channel in which a flow noise is generated during the breathing exercises by an airflow introduced therein, which is recorded by means of a microphone and evaluated by an evaluation unit.

### 2. Description of the Related Art

Such a breathing exercise device is already known from EP 4 175 703 B1. Said document describes a breathing exercise device for treating the respiratory muscles, lung functions and for loosening of mucus in the airways, lungs and/or nasal and pharyngeal cavities of a user. The device consists of a hollow body with a mouth or nose piece and at least one vibrating element inside, which is set into vibration by the user’s inhalation and exhalation. This vibration creates oscillating air pressure fluctuations that contribute to the dilation of the bronchi and the loosening of mucus. The device allows for individual adjustments of airflow and resistance to ensure user-specific therapy.

In the field of breathing exercises and respiratory therapy, it is common to use devices that support users in performing breathing exercises. These devices are designed to strengthen the breathing muscles, increase lung capacity, and improve overall breathing function. It is also known to provide devices that simulate the use of medical devices in order to achieve a training effect with regard to the application of such devices. Known systems typically comprise mechanical devices that are activated by the user’s breathing and provide visual or acoustic feedback to assist the user in performing the exercises correctly. Such devices are often simply constructed and offer basic functions such as measuring breathing volume or breathing rate.

GB 2 563 033 A relates to a measuring device for determining the airflow during inspiration and/or expiration. The device uses a plurality of ridges that protrude into the airflow path to generate at least two sound signals at different resonances. These ridges can be integrated into a plastic body and allow the airflow rate to be displayed without moving parts or the need for regular calibration. The device can be used as a spirometer and offers the possibility to analyze the generated sound signals via a mobile app to provide health feedback or trigger warnings. It is particularly useful for monitoring respiratory diseases such as asthma, as it allows the user to react to changes in breathing capacity.

Furthermore, EP 3 586 740 A1 is known in this field, according to which a lung function device, such as a spirometer, is used to determine, by differential measurement during inspiration and expiration, how much of a substance under consideration remains in the lungs of a user. Additional insights into the vitality parameters of the user in question can be gained by performing prescribed breathing exercises with the device, guided by an associated data processing device, such as a smartphone. Environmental conditions can also be taken into account, either determined by the data processing device itself or obtained from provided sources. Based on the data thus obtained, the system can create and determine the user’s health data, but also provide recommendations for action that can be made available to the user or treating persons. The data obtained can be used to improve the diagnosis and therapy of respiratory diseases such as asthma or chronic obstructive pulmonary disease.

Document US 10,665,132 B2 also describes a training device for teaching the correct use of a pressurized metered-dose inhaler. The device consists of a housing, an air ejection means such as a bellows for ejecting air along an airflow path, and an actuating means for actuating the air ejection means. The airflow path is designed so that, due to a constriction, the air ejection means generates an acoustic signal when expelling air, which imitates the noise made when using a metered-dose inhaler. The device aims to be cost-effective and suitable for home use without the use of propellants, and provides instructions for coordinating inhalation and operation of the medication container. It comprises several features to provide a realistic training experience, including a counter to display the elapsed time since the air ejection means was activated.

According to established techniques, breathing exercise devices are therefore often used in combination with manual or electronic evaluations to monitor the effectiveness of the breathing exercises. However, these systems may be limited in their accuracy and ease of use. For example, some devices only record rough data, which makes a detailed analysis of the user’s breathing patterns difficult. Other systems require manual data entry or separate evaluation by medical professionals, which can make the process time-consuming and prone to errors.

### SUMMARY OF THE INVENTION

Against this background, the object of the present invention is to create a method in which individual training can be carried out orally, tracheally and/or nasally using a simple and user-friendly breathing exercise device, which can be both guided and monitored via an evaluation unit, evaluated based on feedback and adapted based on further environmental parameters.

The object is achieved by a method according to the invention. Meaningful configurations of such a method can be found below.

Insofar as this is provided, a method is provided for recording breathing exercises performed on a breathing exercise device, wherein the breathing exercise device forms a flow channel in which a flow noise is generated during the breathing exercises by an airflow introduced therein, which is recorded by means of a microphone and evaluated by an evaluation unit. According to the invention, such a method is characterized in that the evaluation unit specifies a breathing pattern during the breathing exercise and evaluates the intensity and/or frequency profile and/or duration and/or amplitude of the recorded flow noise and, preferably in the course of an adherence measurement, compares it with the specified breathing pattern and outputs feedback using output means of the evaluation unit.

In other words, a method for performing breathing exercises is described in which a breathing exercise device is used that has a housing which forms at least one flow channel between a breathing opening and a stenosis. When a user breathes into the breathing exercise device, the resulting airflow creates a flow noise that is recorded and evaluated using an evaluation unit.

The evaluation unit determines the intensity and/or vibration frequency profile and/or duration and/or amplitude of the flow noise during the user’s breathing. It records these parameters during a breathing exercise and analyzes and compares them with a predefined breathing pattern provided to the user. The user receives feedback on these parameters and/or feedback on the conformity of the breathing exercise with the specified breathing pattern via output means provided by the evaluation unit, preferably as part of an adherence measurement.

One advantage of this method lies in the precise recording and evaluation of breathing parameters, which allows for targeted feedback to the user, similar to an AI trainer. This can contribute to improving breathing technique and making breathing exercises more effective. Another advantage is the possibility of obtaining detailed information about the breathing process, which can be used for diagnostic and/or therapeutic purposes.

In summary, the present invention relates to a method for providing and optimizing at least one piece of information about the interaction between oral and/or tracheal and/or nasal application of one or more breathing therapy/breathing exercises/relaxation/sleep and inhalation/medication aids and/or medication systems of all kinds, such as metered-dose inhalers with and without counters, mesh/ultrasonic or jet air nebulizers or dry powder inhalers, with the respiratory, lung and movement function, adherence, psychosocial factors and the home and inpatient settings and environments of a user. Specifically, the invention can be used for diagnostics, documentation, monitoring and/or resulting guidance for optimized inhalation, therapy and/or breathing training.

Preferably, such a method may provide that a breathing pattern comprises at least one exhalation, preferably additionally at least one inhalation and/or at least one inhalation pause and/or one exhalation pause.

In general, the method can comprise a training program for correct exhalation in order to quantify lung function as part of a breathing exercise, but also to achieve an exercise effect. This can be supplemented with complete breathing patterns, i.e., the sequence of inhalation, end-inspiratory pause, exhalation and end-expiratory pause, as well as repetitions of the outlined breathing pattern per minute. By performing the test multiple times and recording data over longer periods, monitoring can be achieved and deeper insights into the user’s lung function can be derived.

Furthermore, it can be provided that at least one oscillation unit is received, preferably detachably, in the flow channel in such a way that it is excited by the airflow through the flow channel to acoustically perceptible vibration.

In the specific design of the oscillation unit, it may be provided in particular that the at least one oscillation unit is either a reed unit with at least one reed suspended in a way that allows it to vibrate, or a tube with two inlets, which is forced into an angled or bent pipe in such a way that it always forms a kink between its two inlets.

While in the case of the reed, it begins to vibrate at its resonant frequency in a deep, pleasant tone when an airflow is directed through the at least one flow channel due to the user’s breathing, in the case of the tube, air bubbles are forced through the tube, resulting in a different intensity and/or vibration frequency profile and/or duration and/or amplitude. This allows the evaluation unit to identify which breathing exercise device is being used, so that appropriate advice and instructions can be given.

Additionally, it may be provided that the oscillation unit is designed for mouth breathing and/or nasal breathing and/or tracheal breathing, in particular comprising a reed unit with at least one reed suspended to vibrate and/or a funnel and/or a tube forced into a bent path and/or a ball valve and/or a flap valve.

The method can thus serve to restore functional breathing, i.e., to switch, if necessary, from tracheal breathing to mouth breathing and ultimately to nasal breathing. Nasal breathing allows the incoming air to be filtered, cleaned, humidified and warmed during inhalation. Due to degeneration of the respiratory tract and chronic respiratory diseases, users can often only breathe through their mouths. In the case of a tracheotomy or after abdominal surgery, breathing is only possible via a tracheostomy tube. This breathing is often shallow and not into the abdomen, but mostly into the chest. Such breathing often leads to hyperventilation, i.e., very short, jerky breaths, characterized primarily by a rapid inhalation phase followed by a shortened exhalation phase. However, this breathing technique is not only suitable for chronically ill users, but also for stressed and very tense people in general. This dysfunctional breathing can increase the frequency of infections, even leading to chronic blockages of the upper airways. In addition, growths in the upper respiratory tract, so-called polyp formations, or septal deviations and similar conditions may be present, which inhibit or prevent natural breathing. Chronic respiratory tract blockages may also be present. They all have one thing in common: those affected, approximately 40% of the world’s population, chronically breathe only through their mouths. This exposes them to unnecessary additional risks and infections.

The present system and method are intended to provide a simple remedy here. The design of the interchangeable breathing attachment allows the user to become accustomed to training and/or therapy via the tracheostomy tube or the mouth. The system and method can be used to measure the tonality – it is a sonification of the breathing, the breathing cycle and the breathing technique, i.e., the breaths, their depth and regularity as well as the daily adherence in comparison with the well-being of the user, are recorded, analyzed and, if confirmed, further steps towards the application of oral and/or nasal therapy to resolve the dysfunctional breathing are suggested. If he feels comfortable and has reached a certain level of the type and structure of his breathing, the therapy can begin via the mouth or nose application.

By means of nasal application, it is possible to determine whether there are any blockages of the upper respiratory tract, such as a septal deviation or polyps. If the user is only able to generate an acoustically perceptible signal through one nostril and not through the other, this suggests a complication. In order for the system to recognize this, it is programmed to clearly detect different frequencies and/or airflows.

The system can be equipped with a different airflow for the nose. This airflow is preferably equipped not with air holes, but with a channel that cannot be blocked by secretions. In addition, a distinguishable acoustic signal is transmitted via this other channel. The dual design allows for a clear distinction between breaths during inhalation and exhalation. If no tone is heard, a blockage can be assumed. However, these blockages can also consist solely of secretion retention. To clarify this, the system may first suggest mucolysis via inhalation. If the desired effect is not achieved, the application of decongestant nasal drops can be suggested. If only one nostril opens, it will be a case of unilateral polyp formation or septal deviation. This can then be resolved by administering essential oils or leads to a direct recommendation for an ENT appointment, as further steps will then need to be taken.

In order for a system to detect nasal breathing and any existing primary nasal blockage, it is possible to design the nose piece of the invention in the following way.

Preferably, the evaluation unit can distinguish an inspiratory flow noise from an expiratory flow noise and evaluate the two flow noises separately for comparison with the breathing pattern.

In this context, the differentiation of the parameters means that the evaluation unit is able to analyze the specific values of the flow noises during inhalation and exhalation separately. In particular, it is possible to use a reed that responds in both directions of flow, or to provide a separate reed for each direction of flow, vibrating at its own pitch to make it easy to distinguish between them. The user also receives acoustic feedback.

Different configurations of precise recognition features such as frequency response, amplitudes, oscillations according to pressure and flow, and the corresponding decibels for determining the pressure and flow profile at a precise distance to the mobile device, both during inhalation and exhalation, as well as during the subsequent end-inspiratory and end-expiratory pauses, in which no acoustic signal occurs, but said signal is comprehensible through the sequence of inspiratory and expiratory tonality, can be determined for the precise identification of a breath via mouth and/or nose from various breathing therapy and breathing training devices.

Different flow patterns for nasal, oral, or tracheal application can be distinguished acoustically. Depending on the function of the valves used - such as ball valve, funnel valve and/or flap valve as well as tubes and/or reed valves - different frequency ranges result. The evaluation unit can also automatically detect various casings, which produce characteristic frequency responses, pressure and flow curves, as well as amplitudes and volumes, just like simple valve or stenosis noises.

In this respect, it may additionally be provided that a variable stenosis is assigned to the flow channel, the degree of opening of which is determined by the evaluation unit on the basis of a disturbance noise and/or a change in the frequency profile. It is also possible to work with other variable settings of the breathing exercise device, such as the distance of a funnel and/or a flap valve and/or a combined tube valve. Wherever stenosis is mentioned below, its potential influences also apply here.

This means that the stenosis, which narrows at least one flow channel within the housing of the breathing exercise device, can be adjusted so that its opening size is variable. This adaptability makes it possible to modify the resistance that the airflow experiences in at least one flow channel, which is advantageous for different therapeutic requirements and individual user needs. The degree of opening of the stenosis can be determined by the evaluation unit using a noise, because the narrower the stenosis, the more turbulence occurs in the range of the air exit. This means that the evaluation unit is able to analyze acoustic signals generated by the airflow and interaction with the stenosis and to draw conclusions about the current opening size of the stenosis. This acoustic detection provides precise and continuous monitoring of the stenosis position or the various valve settings, regardless of whether it is a flap, funnel, ball or other valve, without the need for mechanical sensors, thus reducing the complexity and potential susceptibility to error of the system. The frequency response and/or flow noise are affected accordingly.

Additionally, it may be useful if the evaluation unit differentiates the flow noise during breathing through a nose piece, mouthpiece or tracheostomy tube connected to the breathing exercise device, preferably detachably, based on the intensity and/or frequency profile and/or duration and/or amplitude of the recorded flow noise. In principle, it is intended that these different operating modes of the breathing exercise device can be used alternatively.

In specific further training, it may be provided that the evaluation unit specifies a sequence of several breathing cycles and determines how many of these breathing cycles are performed within a breathing exercise, preferably recording training success over a longer period of time and adjusting the training intensity and duration to the progress by the evaluation unit. These stored breathing exercises provide the user with structured instructions for performing the breathing exercises by making them available via the output means of the data processing device. This leads to higher motivation and better therapy success, as the user is continuously challenged and supported through the different exercises and direct feedback. The integration of the output means for providing instructions ensures that the user performs the exercises correctly and that the desired therapeutic effects are achieved.

Furthermore, it may be provided that the evaluation unit performs an initial step at the beginning of a breathing exercise in which the intensity and/or vibration frequency profile and/or duration and/or amplitude of the flow noise of the breathing exercise device as well as ambient noise are recorded, wherein during the subsequent breathing exercise a range around the intensity and/or vibration frequency profile and/or duration and/or amplitude of the flow noise of the respiratory therapy device is isolated using a bandpass filter and ambient noise is filtered out. This data collection is crucial to establishing an accurate baseline for the subsequent analysis. During the breathing exercise, a range around the pitch of the flow noise is isolated using a bandpass filter, thereby filtering out ambient noise. This allows for a clear and undisturbed analysis of the flow noise generated by the user’s breathing. The use of a bandpass filter to isolate the relevant pitch ensures that the evaluation unit accurately records data on the intensity and/or vibration frequency profile and/or duration and/or amplitude of the flow noise during the user’s inspiration and expiration processes, and has to process as little background noise as possible. Filtering out ambient noise minimizes the likelihood of misinterpretations or disturbances, leading to more reliable and effective breathing therapy.

In particular, it may be provided that the evaluation unit checks the tone signals received during the initial step against the sound pattern of an ultrasonic nebulizer or other medication nebulizers. Flow or trigger noises from the medication containers can be detected, recorded and analyzed. The airflow during inhalation through medication containers can also be precisely determined, recorded, and, if necessary, trained or corrected in advance by measuring amplitude, frequency, period, and intensity. Thus, nebulization of any type of medication, with or without combination with breathing therapy, can be used to more accurately monitor the interaction of infections, hospital stays, and the need for medication in combination with the aforementioned aids such as breathing exercises and breathing therapy, as well as relaxation, inhalation aids, and sleep aids, and, if necessary, to optimize their combination.

Furthermore, the evaluation unit can either be assigned in a structurally integrated manner to the breathing exercise device or can be assigned to a data processing device, wherein electronic communication means are assigned to the breathing exercise device for data exchange with the data processing device, wherein at least data for identifying the type of breathing exercise device can be transmitted by the breathing exercise device to the data processing device via the electronic communication means.

Combined with wearables and/or sleep systems and/or ventilation systems, the system according to the invention can incorporate additional data into the evaluation. The most important data here are heart rate, oxygen saturation, and blood pressure. Lowering the breathing rate leads to a decrease in the heart rate, and depending on the design of the breathing cycle, also to a change in gas exchange. This in turn affects blood pressure. Accordingly, these parameters can be optimally influenced through regular training and/or therapy. Furthermore, the reduction in breathing rate has a calming effect on the user. Provided it is used regularly, this can make it easier for the person to fall asleep.

A further development of this case may provide that breathing exercises with different sequences, in particular with regard to intensity and/or vibration frequency profile and/or duration and/or amplitude of the flow noises, settings and/or tone sequences, are stored on the data processing device and instructions for carrying out these breathing exercises, preferably including embedding in music and video data to accompany the breathing exercises, are provided via the output means of the evaluation unit. This can help the user to further adjust their breathing rhythm individually for relaxation, sleep or meditation.

It may also be possible to integrate a daily and/or weekly schedule, or even longer-term therapy plans, into the data processing device. This plan allows the data processing device to issue reminders to the user at predetermined times via its output means. Integrating a daily and/or weekly schedule into the data processing device offers several advantages. Firstly, it supports the regular performance of breathing exercises by reminding the user to perform the exercises at set times. This promotes adherence to the therapy plan and can lead to better therapy outcomes. Secondly, the plan can be individually tailored to the needs and daily routine of the user, which increases the flexibility and ease of use of the method. Finally, integrating such a plan can also increase the user’s motivation, as the reminders continuously remind them of the importance and benefits of the breathing exercises.

Preferably, the data processing device may also be assigned means for data input and/or for recording and/or retrieving data from a database and/or for automatically suggesting or specifying data, wherein the data comprise

user groups and/or pre-existing conditions of a user, in particular degrees of bronchial obstruction and/or frequencies of exacerbations, comorbidities and/or symptom burdens,

and/or parameters of inpatient and outpatient rehabilitation measures and/or inpatient and outpatient therapeutic and/or medical care or medication of a user,

and/or location and/or calendar data, event data or health warnings from public authorities,

and/or location data, and preferably also anonymized user data from other users.

Particularly relevant here are data on the degree of bronchial obstruction, the frequency of exacerbations, comorbidities and symptom burden. Integrating the means for data input and collection offers several advantages. Firstly, it allows for a more precise and individualized adjustment of the breathing exercises to the specific needs and health conditions of the users. This is particularly important for users with chronic respiratory diseases, as the therapy can thus be tailored to the respective degrees of bronchial obstruction and the frequency of exacerbations. The collected data can also be used to identify patterns and trends that indicate a deterioration in health, so that countermeasures can be taken early.

It is considered particularly advantageous if the data comprises user groupings and/or pre-existing conditions of the user, in particular degrees of bronchial obstruction and/or frequencies of exacerbations, comorbidities and/or symptom burdens, parameters of hospital stays or medication, location and/or calendar data, event data or health warnings from public authorities, and/or location data, as well as preferably also anonymized user data of other users. In such a case, the user can be instructed according to procedure on when, how (nasal/oral/tracheal) and where breathing exercises and/or breathing therapy with and without inhalation or medication administration should be carried out more frequently, using oscillating breathing therapy and training devices with and without humidified inhalation or other medication administration via mesh nebulizers, metered-dose inhalers and/or dry powder inhalers, etc. This can be done in such a case based on the interaction of location data, calendar data and data from health authorities and institutions. This depends, for example, on whether there is an increased amount of time spent indoors, whether there is increased activity and contact with many people in a restaurant or while shopping, whether it is, for example, carnival or Christmas time and increased gathering activity is to be expected. The location is also important in order to take local customs and seasons into account. With regard to health data, in the case of known flu epidemics and other data on the spread and thus an increased possibility of exposure to viruses and bacteria, a reaction can be taken and additional caution and preventive therapeutic measures can be recommended.

Furthermore, the linking of location data is relevant for the beginning of the heating season. Then people spend more time indoors. The risk of infection also increases, among other things, through contact with small children. Infection hotspots can be avoided through shared location information, tracking, calendar and/or event data from the Internet. Relevant insights can be derived from the data, which are continuously adapted and refined through evaluation data. The user may be asked by the evaluation unit to implement humidification measures, in particular regular ventilation or alternatively the use of humidifying nasal sprays, humidifying inhalation, or the use of, for example, corticosteroids. However, even with tracheal application, a certain routine can be established by comparing hospital data and a corresponding frequency of use can be suggested. To improve sleep hygiene, in particular nasal breathing, this can be applied more frequently before sleeping, and the user can then be guided to better sleep hygiene through a breathing pattern of meditation or, to a lesser extent, relaxation, preferably via purely acoustic and not visual instructions. A comparison with the length and intensity of sleep and a corresponding suggestion for the next night are considered advantageous here, since physical regeneration is particularly promoted during sleep.

It is particularly advantageous if the data processing device is assigned an artificial intelligence that evaluates entered and/or recorded data and/or parameters and makes suggestions for breathing exercises or other data and/or parameters. This artificial intelligence evaluates entered and/or recorded data and makes suggestions for further breathing exercises. The integration of artificial intelligence into the data processing device allows for advanced analysis of the collected data. It can identify patterns and trends in breathing data and, based on this, make personalized suggestions for further breathing exercises. This offers the user a tailored therapy based on their individual progress and needs. One advantage of this embodiment is the possibility of increasing the efficiency and effectiveness of the breathing exercises by having the artificial intelligence continuously learn and adapt to the specific requirements of the user. Another advantage is the improvement of user motivation and retention through the provision of personalized exercise suggestions and real-time feedback. Artificial intelligence helps to optimize therapy outcomes and promote the long-term health and well-being of the user. This makes individualized care possible even with fewer staff.

In an advantageous design, it may also be provided that the feedback comprises a point system, a progress visualization, or at least one gamification element. This provides the user with a motivating and interactive user experience. The point system allows the user to collect points for correctly performed breathing exercises, resulting in a gamified and rewarding experience. This can increase the user’s motivation and engagement, as they can make progress visible by collecting points. A progress visualization provides the user with a graphical representation of their progress over time. This can be done in the form of charts, bars or other visual indicators that give the user clear and understandable feedback about their performance and improvements. A gamification element can comprise various game-like elements, such as reaching levels, unlocking rewards, or achieving milestones. These playful elements can make training more entertaining and engaging, which in turn can promote compliance and long-term use of the device. Furthermore, the use of such interactive and motivating elements can help improve therapy outcomes by encouraging the user to become more involved in the therapy process.

Furthermore, it may be stipulated that the data processing device is a laptop, a tablet, a smartphone, a wearable, a lung function device, a continuous positive airway pressure (CPAP) machine, a cough assist device and/or a cough detector.

Finally, it seems sensible if the output means comprise a screen and/or a speaker, such as headphones. This allows visual, acoustic, and haptic signals to be used to communicate with the user.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the Drawings:

FIG. 1 shows an embodiment of a breathing exercise device and a data processing device used to perform breathing exercises via a mouthpiece in a schematic sectional view;

FIG. 2 shows an embodiment of a breathing exercise device with a nasal piece, wherein the breathing exercise device is connected to a data processing device;

FIG. 3 shows an embodiment of a breathing exercise device with a housing and a rotatable stenosis; and

FIG. 4 shows another embodiment of a breathing exercise device with an oscillation unit in the form of a tube.

### DETAILED DESCRIPTION OF PREFERRED EMBODIMENTS

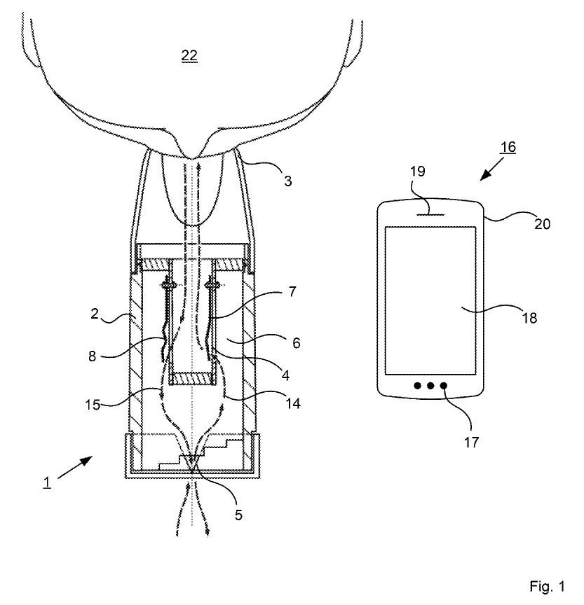

FIG. 1 shows a schematic representation of a system for performing breathing exercises, consisting of a breathing exercise device 1 and a data processing device 16. The breathing exercise device 1 comprises a housing 2 that forms a flow channel 6 between a mouthpiece 3 and a stenosis 5. Within the flow channel 6, two reeds 7 and 8 are arranged which are suspended so as to be able to vibrate and which can be set into vibration by an airflow. A first reed 7 is provided for the inspiration process 14 and a second reed 8 for the expiration process 15, so that the reeds 7 and 8 can be operated at different pitches. This results in a characteristic tone sequence for the user 22.

The data processing device 16 is a conventional smartphone with a software product that performs the present method. It is equipped with a microphone 17, an evaluation unit 20, such as the program and the processors, registers and memory required for it, as well as output means in the form of a screen 18 and a speaker 19. As part of a breathing exercise, the smartphone with microphone 17 is positioned so that it records the vibrations of the reeds 7 and 8, which are generated by the breath of the user 22. The recorded data are forwarded within the data processing device 16 to the evaluation unit 20, which analyzes and records the vibration frequency and intensity of the vibrations during the inspiratory processes 14 and expiratory processes 15, as well as the duration of the inspiratory and expiratory pauses in between. From this, the evaluation unit 20 determines a breathing pattern, which is compared with target specifications and evaluated taking into account the other data entered into the data processing device 16 by the user 22, as well as additional data from databases, e.g. for events. This allows consideration of whether the user 22 is at the location and time of an event with many people, so that in this case different measures may be recommended due to the risk of infection than in other locations or at other times.

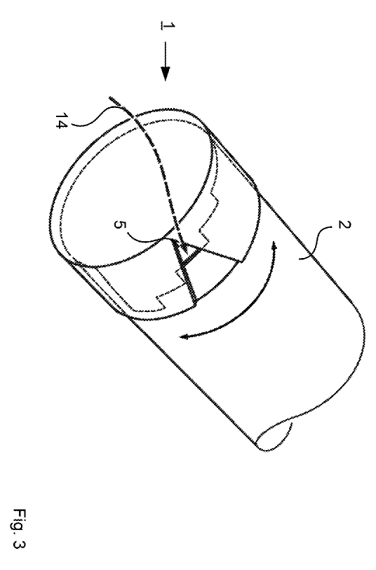

The stenosis 5 in the flow channel 6 is variable in its opening size, which makes it possible to adjust the resistance of the airflow. This is shown in detail in FIG. 3. The evaluation unit 20 can use a noise generated by air turbulence at the stenosis 5 to detect the degree of opening of the stenosis 5 and give feedback to the user 22 via the output means 18 and 19, which suggests an adjustment of the stenosis 5 if this results from the evaluation of the breathing exercises. Insofar as the stenosis 5 is electronically adjustable, such an adjustment can also be made directly at the instigation of the evaluation unit 20 via an actuator not shown here.

The evaluation unit 20 distinguishes the vibration frequencies of the vibrations during the inspiration process 14 and the expiration process 15 in order to measure a breathing pattern of the user 22. In addition, the duration of the vibrations and the rest phases between breathing processes are recorded and evaluated in order to determine the regularity of the breathing cycles.

At the beginning of a breathing exercise, the evaluation unit 20 performs an initial step in which the pitch of the reeds 7 and 8 as well as ambient noises are recorded. A bandpass filter isolates a range around the pitch of reeds 7 and 8 during the breathing exercise to block out ambient noise. Here, the breathing exercise device 1 is equipped with a mouthpiece 3 designed for mouth breathing, to which reeds 7 and 8 are assigned in a reed unit 4, which are suitable for mouth breathing, in particular requiring a stronger airflow to be stimulated to vibrate. The reed unit 4 can be connected as a whole to the mouthpiece 3, so that a change to a different breathing attachment, such as a nose piece 21, also leads to a different configuration of reeds 7 and 8. Alternatively, the reed unit 4 can also be removed separately and replaced as needed.

The breathing exercise device 1 can also be equipped with electronic communication means in an alternative configuration, which is not shown here, allowing data exchange with the data processing device 16. This comprises, in particular, data for identifying the type of breathing exercise device 1 and the pitches of the reeds 7 and 8. Electronic recording and transmission of the stenosis settings 5 to the data processing device 16 is also possible. The complete integration of the evaluation unit into the breathing exercise device can also take place in this context.

Various breathing exercises with different sequences are stored on the data processing device 16. These exercises can vary with regard to the duration of the inspiratory processes 14, the duration of the expiratory processes 15, the pauses in between, the number of repetitions, the specified vibration frequency, settings and/or tone sequences, and provide the user 22 with instructions for performance via the output means (screen 18 and speaker 19). During the breathing exercises, the data processing device 16 performs an adherence measurement, in which the measured pitches of the reeds 7 and 8 are compared with the specifications of the breathing exercises, also over time.

The data processing device 16 can also contain a daily and/or weekly schedule that reminds the user 22 to perform the breathing exercises at predetermined times. In addition, there are means available for data input and/or for recording or retrieving data from databases, which comprise user groups and/or pre-existing conditions as well as other data if applicable.

An artificial intelligence in the data processing device 16 can evaluate the entered, recorded and retrieved data and make suggestions for further breathing exercises. The system can also comprise a point system, progress visualization, or gamification elements to provide the user 22 with feedback on training and therapy successes.

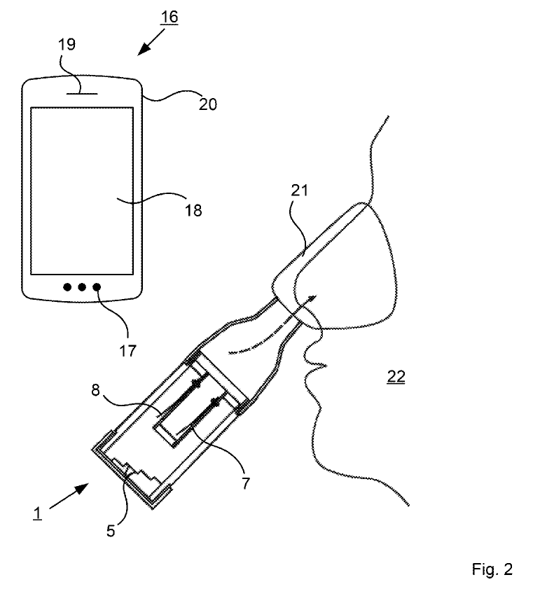

In an alternative embodiment according to FIG. 2, the breathing attachment can be designed as a nose piece 21. The data processing device 16 can be designed in the form of a smartphone or a wearable, or can be fully integrated into the breathing exercise device 1.

FIG. 3 shows a perspective view of the lower end of a breathing exercise device 1. The housing 2 of the device forms a flow channel that runs between a breathing opening and a stenosis 5. The stenosis 5 is positioned within the flow channel 6 and its opening size can be changed to regulate the airflow, here an inspiration process 14.

The housing 2 is designed to allow an airflow through the flow channel 6 when a user 22 breathes through a breathing opening with a mouthpiece 3 or a nose piece 21, or also through a tracheostomy tube. The stenosis 5 is arranged in such a way that it causes a narrowing of the flow channel 6, thereby increasing the speed of the airflow and thus exciting a vibration of the reeds 7 or 8 located in the flow channel 6.

The stenosis 5 can be adjusted mechanically or electronically to change the degree of opening. In this case, one possible modification is the mutual rotation of a lower slotted element against an upper, stepped element. By rotating the slotted part to the right, the stenosis 5 is opened further; by rotating the slotted part to the left, it is closed further. This adjustment allows for fine-tuning of breathing resistance, which is important for performing specific breathing exercises. The opening size of the stenosis 5 can be changed not only by a rotational movement but also by another suitable mechanism.

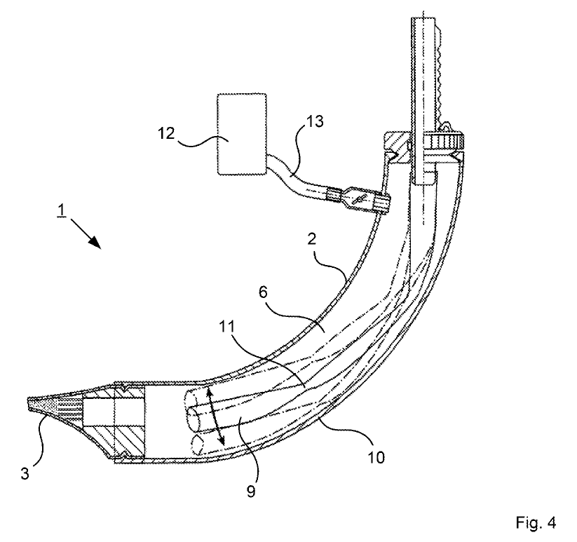

FIG. 4 shows a further embodiment of a breathing exercise device 1, which works with a tube 9 instead of the reeds shown so far. Said tube is confined within a bent pipe 10, so that the tube 9 always has at least one kink 11 in the pipe 10. When inhaling through the mouthpiece 3, a user can draw in air through the tube 9, causing the kink 11 to move through the tube 9 towards the breathing attachment 3 until an air bubble can escape from the free end of the tube 9. As with the reeds, this creates an oscillation that can be recorded and evaluated by a microphone of a data processing device.

If required, in addition to the actual breathing exercise, aromatherapy or nebulization of active ingredients can also take place in the housing 2, so that the breathing air mixed with it is inhaled by the user 22. For this purpose, an inhalation device 12 is connected to the housing 2 via a connecting tube 13 such that the connecting tube 13 opens into the flow channel 6. In addition to the air that enters the breathing exercise device 1 through the tube, the active ingredient held in the inhalation device 12 is also added, which can take effect when inhaled by the user 22. Such a measure can be taken as needed by the evaluation unit 20 of the data processing device 16 if the results of previous breathing exercises or the data situation so require.

A method is described above in which individual training can be carried out orally, tracheally and/or nasally using a simple and user-friendly breathing exercise device, which can be both guided and monitored via an evaluation unit, evaluated based on feedback and adapted based on further environmental parameters.

Although only a few embodiments of the present invention have been shown and described, it is to be understood that many changes and modifications may be made thereunto without departing from the spirit and scope of the invention.

### LIST OF REFERENCE SIGNS

1 Breathing exercise device

2 Housing

3 Mouthpiece

4 Reed unit

5 Stenosis

6 Flow channel

7 First reed

8 Second reed

9 Tube

10 Pipeline

11 Kink

12 Inhalation device

13 Connecting tube

14 Inspiration process

15 Expiration process

16 Data processing device

17 Microphone

18 Screen

19 Speaker

20 Evaluation unit

21 Nose piece

22 User

What is claimed is:

1. A method for recording breathing exercises performed on a breathing exercise device (1), wherein the breathing exercise device (1) forms a flow channel (6) in which a flow noise is generated during the breathing exercises by an airflow introduced therein, which is recorded by means of a microphone (17) and evaluated by an evaluation unit (20),
wherein the evaluation unit (20) specifies a breathing pattern during the breathing exercise and evaluates the intensity and/or frequency profile and/or duration and/or amplitude of the recorded flow noise and, preferably in the course of an adherence measurement, compares it with the specified breathing pattern and outputs feedback using output means of the evaluation unit (20).

2. The method according to claim 1, wherein a breathing pattern comprises at least one exhalation, preferably additionally at least one inhalation and/or at least one inhalation pause and/or one exhalation pause.

3. The method according to claim 1, wherein at least one oscillation unit is received, preferably detachably, in the flow channel (6) in such a way that it is excited by the airflow through the flow channel (6) to acoustically perceptible vibration.

4. The method according to claim 3, wherein the oscillation unit is designed for mouth breathing and/or nasal breathing and/or tracheal breathing, in particular comprising a reed unit (4) with at least one reed (7, 8) suspended to vibrate and/or a funnel and/or a tube (9) forced into a bent path and/or a ball valve and/or a flap valve.

5. The method according to claim 1, wherein the evaluation unit distinguishes an inspiratory flow noise from an expiratory flow noise and evaluates the two flow noises separately for comparison with the breathing pattern.

6. The method according to claim 1, wherein a variable stenosis (5) is assigned to the flow channel (6), the degree of opening of which is determined by the evaluation unit (20) on the basis of a disturbance noise and/or a change in the frequency profile.

7. The method according to claim 1, wherein the evaluation unit (20) differentiates the flow noise during breathing through a nose piece (21), mouthpiece (3) or tracheostomy tube connected to the breathing exercise device (1), preferably detachably, based on the intensity and/or frequency profile and/or duration and/or amplitude of the recorded flow noise.

8. The method according to claim 1, wherein the evaluation unit (20) specifies a sequence of several breathing cycles and determines how many of these breathing cycles are performed within a breathing exercise, preferably recording training success over a longer period of time and adjusting the training intensity and duration to the progress by the evaluation unit.

9. The method according to claim 1, wherein the evaluation unit (20) performs an initial step at the beginning of a breathing exercise in which the intensity and/or vibration frequency profile and/or duration and/or amplitude of the flow noise of the breathing exercise device (1) as well as ambient noise are recorded, wherein during the subsequent breathing exercise a range around the intensity and/or vibration frequency profile and/or duration and/or amplitude of the flow noise of the breathing exercise device (1) is isolated using a bandpass filter and ambient noise is filtered out.

10. The method according to claim 9, wherein the evaluation unit (20) checks the tone signals received during the initial step against the sound pattern of an ultrasonic nebulizer or other medication nebulizers.

11. The method according to claim 1, wherein the evaluation unit (20) is assigned in a structurally integrated manner to the breathing exercise device (1).

12. The method according to claim 1, wherein the evaluation unit (20) is assigned to a data processing device (16), and an electronic communicator is assigned to the breathing exercise device (1) for data exchange with the data processing device (16), wherein at least data for identifying the type of breathing exercise device (1) can be transmitted by the breathing exercise device (1) to the data processing device (16) via the electronic communicator.

13. The method according to claim 12, wherein breathing exercises with different sequences, in particular with regard to intensity and/or vibration frequency profile and/or duration and/or amplitude of the flow noises, settings and/or tone sequences, are stored on the data processing device (16) and instructions for carrying out these breathing exercises, preferably including embedding in music and video data to accompany the breathing exercises, are provided via the output means of the evaluation unit.

14. The method according to claim 12, wherein the data processing device (20) is assigned means for data input and/or for recording and/or retrieving data from a database and/or for automatically suggesting or specifying data, wherein the data comprise
user groups and/or pre-existing conditions of a user, in particular degrees of bronchial obstruction and/or frequencies of exacerbations, comorbidities and/or symptom burdens,
and/or parameters of inpatient and outpatient rehabilitation measures and/or inpatient and outpatient therapeutic and/or medical care or medication of a user (22),
and/or location and/or calendar data, event data or health warnings from public authorities,
and/or location data, and preferably also anonymized user data from other users.

15. The method according to claim 14, wherein the data processing device (16) is assigned an artificial intelligence that evaluates entered and/or recorded data and/or parameters and makes suggestions for breathing exercises or other data and/or parameters.

16. The method according to claim 12, wherein the feedback comprises a point system, a progress visualization or at least one gamification element.

17. The method according to claim 12, wherein the data processing device (16) is a laptop, a tablet, a smartphone, a wearable, a lung function device, a continuous positive airway pressure (CPAP) machine, a cough assist device and/or a cough detector.

18. The method according to claim 1, wherein the output means comprise a screen (18) and/or a speaker (19), such as headphones.
